(19) United States
(12) Patent Application Publication
(10) Pub. No.: US 2011/0178367 A1
(43) Pub. Date: Jul. 21, 2011

(54) ENDOSCOPIC DEVICE WITH END EFFECTOR MECHANISM

(75) Inventor: Mehran Anvari, Hamilton (CA)

(73) Assignee: MCMASTER UNIVERSITY, Hamilton, ON (CA)

(21) Appl. No.: 13/003,485

(22) PCT Filed: Jul. 10, 2009
(86) PCT No.: PCT/CA2009/000967

§ 371 Date: Apr. 8, 2011

Related U.S. Application Data
(60) Provisional application No. 61/079,598, filed on Jul. 10, 2008.

(51) Int. Cl. A61B 17/94

(52) U.S. Cl. 600/106; 600/104

(57) ABSTRACT

An endoscopic device and associated method of use. The endoscopic device includes a tubular portion and an end effector mechanism for mounting at an end of the tubular portion. The end effector mechanism includes a casing defining a cavity open to an exterior of the casing. A manipulator member is nested within the cavity of the casing and extendable to protrude to the exterior of the casing. The manipulator member includes a plurality of serially coupled manipulator segments and ending with a last manipulator segment. Each manipulator segment is pivotally connected to an adjacent manipulator segment. A surgical end effector tool such as a gripper or cutter is mounted at the last manipulator segment. A mechanical control may be located within the casing and used for mechanically controlling of the manipulator member. The endoscopic device may operate from a single port or natural orifice of a patient.

### CROSS-REFERENCE TO RELATED APPLICATIONS

The present application claims priority from U.S. Provisional Application No. 61/079,598 filed Jul. 10, 2008, the contents of which are herein incorporated by reference.

### FIELD

Example embodiments described herein relate to surgical devices, and in particular to an end effector mechanism for use in endoscopic surgery.

### BACKGROUND

In some conventional endoscopic procedures, multiple incision sites are made for access of surgical instruments into the abdominal cavity. Such procedures require surgical instruments to enter each incision site in order to perform the desired procedures within the cavity. The number and size of the incisions may result in slower recovery and additional pain to a patient. For example, some conventional endoscopic procedures require access to all quadrants of the abdomen. Some conventional systems may be incapable of simultaneously working in all four quadrants of the abdomen unless repositioned manually. Repositioning of the patient or system could be time consuming and impractical in a time-sensitive surgical environment.

### SUMMARY

In one aspect, there is provided an end effector mechanism for mounting to an end of an endoscopic device. The end effector mechanism includes a casing defining a cavity open to an exterior of the casing. A manipulator member is nested within the cavity of the casing and extendable to protrude to the exterior of the casing. The manipulator member includes a plurality of serially coupled manipulator segments and ending with a last manipulator segment. Each manipulator segment is pivotally connected to an adjacent manipulator segment. A surgical end effector tool such as a gripper or cutter is mounted at the last manipulator segment. A mechanical control may be located within the casing and used for mechanically controlling of the manipulator member.

### BRIEF DESCRIPTION OF THE DRAWINGS

Reference will now be made, by way of example, to the accompanying drawings which show example embodiments, and in which:

FIG. 1 shows a perspective diagrammatic view of a robotic surgical system in accordance with an example embodiment;

FIG. 2 shows a side diagrammatic view of an endoscopic device to be used in the system of FIG. 1;

FIG. 3 shows a front view of an end effector mechanism mounted at an end of the endoscopic device of FIG. 2;

FIG. 4 shows a side partial cross-sectional view of the end effector mechanism of FIG. 3;

FIG. 5 shows a side view of a manipulator member having a surgical end effector tool mounted thereon to be used in the end effector mechanism of FIG. 3;

FIG. 6 shows a partial side diagrammatic view of a manipulator member having a camera device mounted thereon to be used in the end effector mechanism of FIG. 3;

FIG. 7 shows a perspective of the end effector mechanism of FIG. 3 in a nested mode of operation;

FIG. 8 shows a perspective of the end effector mechanism of FIG. 3 in an extended mode of operation; and

FIG. 9 shows a perspective of the end effector mechanism of FIG. 3 in an operating mode of operation.

Similar reference numerals may be used in different figures to denote similar components.

### DETAILED DESCRIPTION OF EXAMPLE EMBODIMENTS

Some conventional endoscopic procedures rely on endoscopes of various sizes and deploy small cutters and forceps through an existing channel of the endoscope to access the surgical site. A difficulty with such a procedure is that the cutters and forceps require lead ends which must be threaded through the channel and which may be cumbersome to manipulate, especially in a time-sensitive surgical environment. Similarly, since the lead lines need to be at least a length of the endoscope, much surgical-grade material may be required for such procedures.

Some other endoscopic procedures are performed through a single incision site, for example either through a single access port such as transabdominal or through natural orifices such as the vagina. One advantage of such techniques is reduced number of incision sites and the ability to perform the procedure with only one point of access. However, a tradeoff in existing techniques is lack of precision and reduced degrees of freedom for the practitioner using such techniques. In conventional laparoscopic or endoscopic tools the degrees of freedom therefore becomes restricted or limited.

It would be advantageous to reduce or limit of the number and size of incisions to result in faster recovery and reduced pain to the patient.

It would also be advantageous to provide a surgical device which may be manipulated with additional degrees of freedom.

In example embodiments, there is generally provided an endoscopic device including a tubular portion and an end effector mechanism for mounting at an end of the tubular portion. The end effector mechanism includes a casing defining a cavity open to an exterior of the casing. A manipulator member is stowed or nested within the cavity of the casing and extendable to protrude to the exterior of the casing. The manipulator member includes a plurality of serially coupled manipulator segments and ending with a last manipulator segment. Each manipulator segment is pivotally connected to an adjacent manipulator segment. A surgical end effector tool such as a gripper or cutter is mounted at the last manipulator segment. A mechanical control may be located within the casing and used for mechanically controlling of the manipulator member.

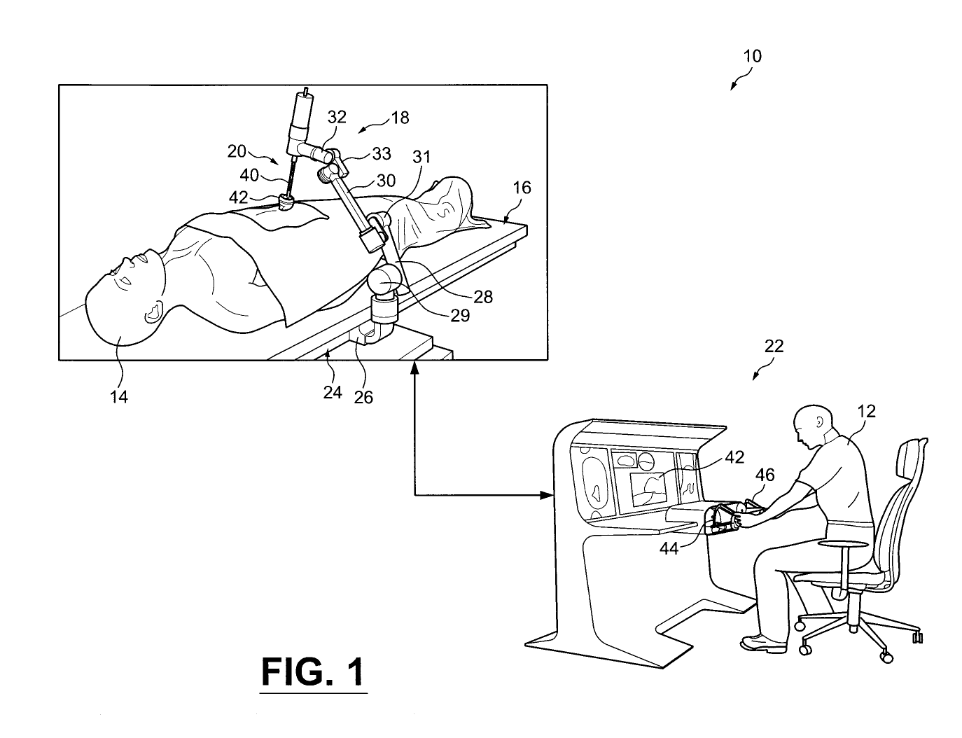

Reference is now made to FIG. 1, which shows a perspective diagrammatic view of a robotic surgical system 10 in accordance with an example embodiment. The system 10 is configured for assisting a practitioner 12 in performing endoscopic surgery within a single access point such as an access port 42 inserted at the abdomen of a patient 14. A surgical bed 16 is shown which includes a platform with a generally flat surface for supporting of the patient 14 and having longitudinal and transverse dimensions. Also shown is a mounting mechanism 18 attached to the surgical bed 16 and an endoscopic device 20 extending from the mounting mechanism 18 (inserted and shown partially covered by the patient 14). The system 10 includes a workstation 22 which is in communication with the mounting member 18 and the endoscopic device 20, for control and operation of same.

The surgical bed 16 has defined therein a track 24 in the form of a groove which runs along a length at one or all edges of the surgical bed 16. The mounting mechanism 18 includes a mounting base 26 which has a portion mounted onto the track 24 and is therefore moveable with respect to the surgical bed 16, along the track 24. This for example assists the mounting mechanism 18 in moving translationally with respect to the surgical bed 16 for accessing different abdominal regions of the patient 14.

The mounting mechanism 18 further includes a plurality of serially coupled mounting segments, each mounting segment pivotally connected to an adjacent mounting segment. As shown, a first mounting segment 28 is mounted via a pivot joint 29 to the mounting base 26 and rotatable in a plane generally in the transverse direction of the surgical bed 16. A second mounting segment 30 is mounted to the first mounting segment 28 via pivot joint 31, and as shown is also rotatable in the transverse direction. A third (and last) mounting segment 32 is mounted to the segment mounting segment 30 via pivot joint 33 and rotatable in the transverse direction. As can be appreciated other mounting segments may be added which may be rotatable in other planes, such as the longitudinal direction of the surgical bed 16 or may even be axially rotatable. The mounting mechanism 18 as illustrated in FIG. 1 therefore includes one degree of freedom from the mounting base 26 and three degrees of freedom from the mounting segments 32, resulting in four degrees of freedom for operation.

Referring still to FIG. 1, the endoscopic device 20 includes a tubular portion 40 having a proximal end mounted to the third mounting segment 32 and a distal end for entering within the patient 14. As shown, the patient 14 may be provided with the access port 42 for insertion of the tubular portion 40. A natural orifice may alternatively be used for access to the patient 14 such as trans-anal, trans-vaginal, or trans-esophagus.

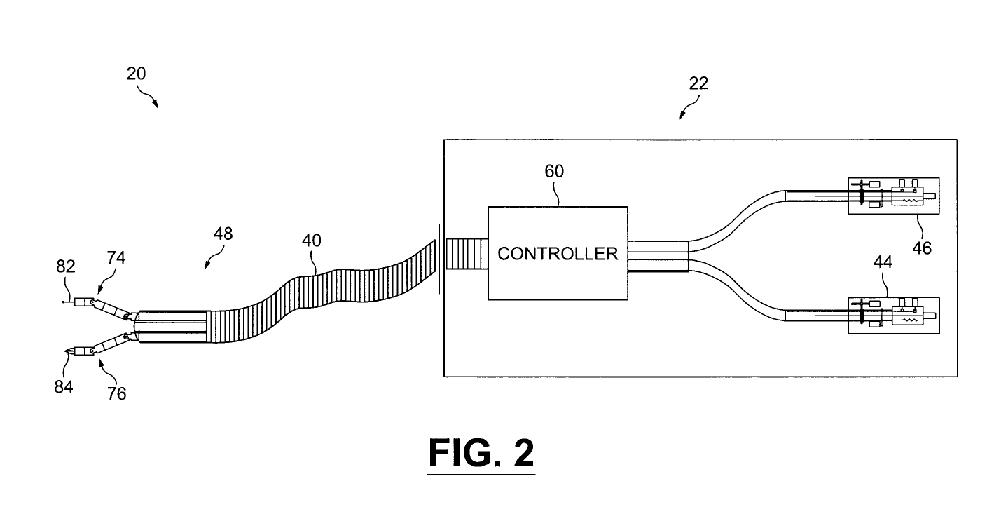

Reference is now made to FIG. 2, which shows a side diagrammatic view of the endoscopic device 20. As shown in FIG. 2, the tubular portion 40 includes a snake-like catheter with a number of snake segments connected via joints which can form bends between the snake segments. Each of the snake segments is individually controllable via left and right hand-activated controllers 44, 46 to bend at the joints which allows for navigation inside of the patient 14. In other example embodiments, the endoscopic device 20 may include components from existing conventional endoscopes, as would be appreciated by those skilled in the art.

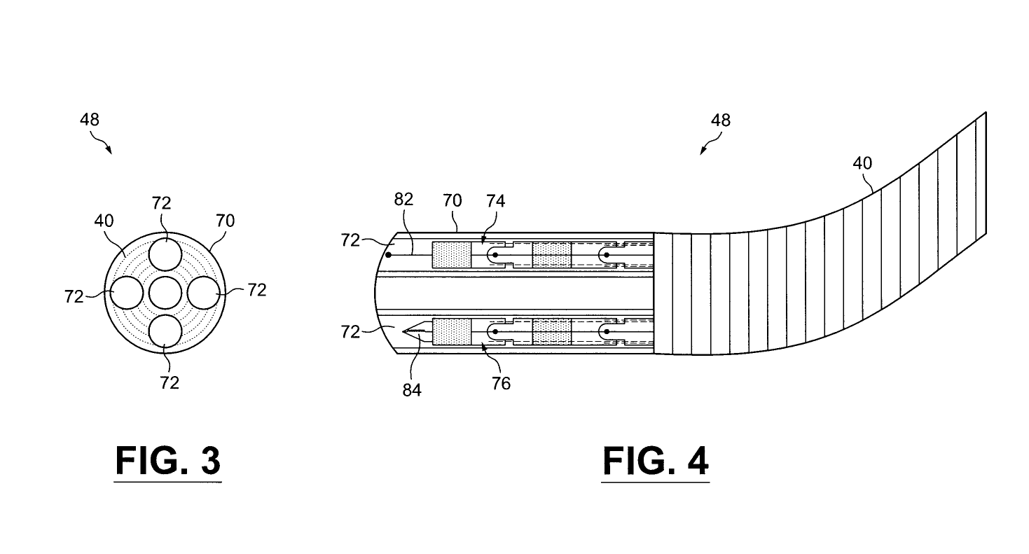

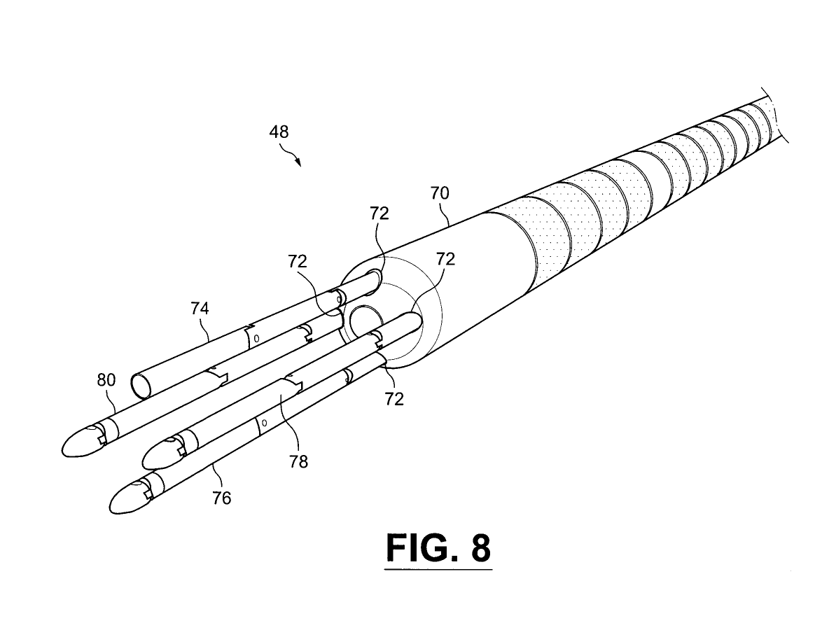

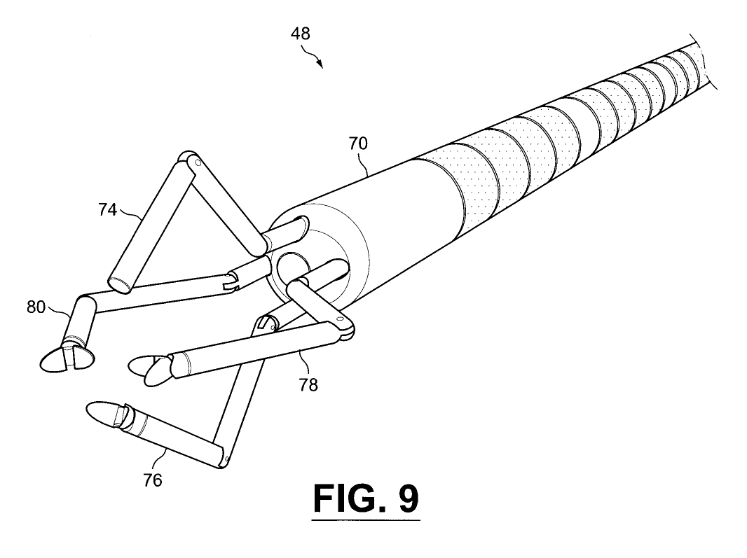

The end effector mechanism 48 will now be described in greater detail. As best shown and briefly referring to FIGS. 8 to 9, the end effector mechanism 48 includes a housing having a casing 70, the casing 70 defining separately-defined cavities or channels 72 open to an exterior of the casing 70. For example, four channels 72 may be separately defined, as shown. Also shown is four manipulator members 74, 76, 78, 80 which are each located within one of the four channels 72. Each manipulator member 74, 76, 78, 80 has a proximal end and a distal end, wherein each of the distal ends are normally stowed or nested within the channels 72. Reference is now made to FIGS. 3 and 4, wherein FIG. 3 shows a front view of the end effector mechanism 48 located at the distal end of the tubular portion 40, and FIG. 4 shows a side partial cross-section of the end effector mechanism 48. A detector such as a camera device 82 is mounted on the manipulator member 74. A surgical end effector tool such as a gripper 84 is mounted on the manipulator member 76. The casing 70 may have a generally circular axial cross-section and be hemispherical shaped, and may for example have a diameter of 20 mm (millimetres) or less. The channels 72 may be shaped to snuggly receive a cross-sectional shape of the manipulator members 74, 76, 78, 80. For example, each of the channels 72 may be 3.1 mm in diameter while the manipulator members 74, 76, 78, 80 may each be 3 mm in diameter. Such a configuration may assist in pushing the casing 70 through voids in tissues and steer to the intended surgical site.

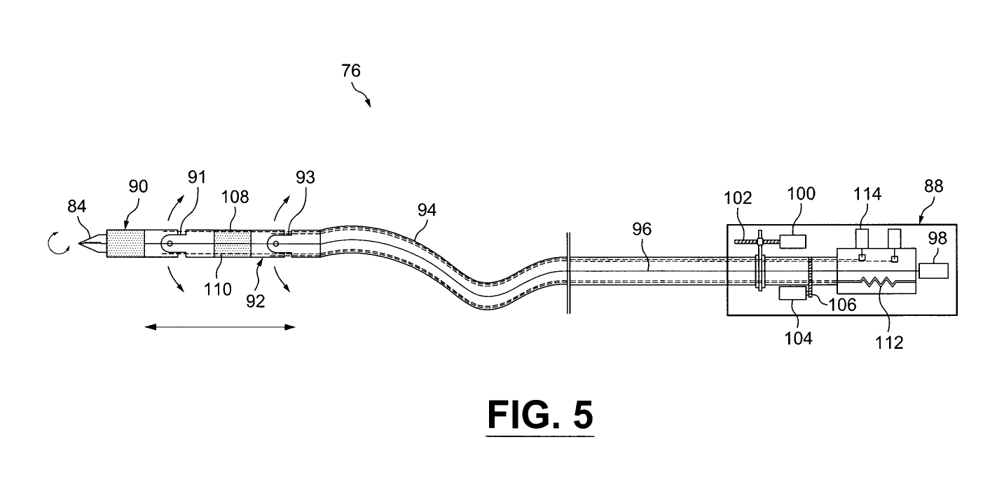

Reference is now made to FIG. 5 which shows the manipulator member 76 having the gripper 84 mounted on the distal end thereof. Generally, the manipulator member 76 is configured to have additional degrees of freedom while extended from the casing 70, for use at a surgical site. A mechanical control such as drive box 88 mechanically controls the operation of the manipulator member 76. The drive box 88 may for example be located within the casing 70 and controllable via communication with the workstation 22 (FIG. 1), which is located remotely to the casing 70. The gripper 84 is operated via a tool drive cable 96 which is located within the flex shaft 94 and runs along a length of the flex shaft 94. The gripper 84 is controlled or actuated by a tool motor 98 which pulls or releases the drive tool cable 96 to close or open the gripper 84, respectively, as can be appreciated by those skilled in the art.

The manipulator member 76 includes a plurality of serially coupled manipulator segments 90, 92, each manipulator segment pivotally connected to an adjacent manipulator segment. As shown, manipulator segment 90 is mounted via a pivot joint 91 to manipulator segment 92, which is subsequently mounted to a flex shaft 94 via pivot joint 93. The gripper 84 is mounted to manipulator segment 90. The pivot joints 91, 93 are each controlled by a pair of cables 108, 110 which actuate the joints. When one cable is in tension and the other is relaxed the pivot joint 91 will bend (similar to how a human joint operates). To achieve opposite motion the other cable of the pair of cables 108, 110 is in tension while the one cable is relaxed. This is controlled by the controller (FIG. 2) so that suitable opposing tension and relaxation is achieved for operation. Bias members such as springs 112 (only one shown) on the proximal end of the drive cables 108, 110 provide a reaction force by constantly taking up slack on the opposing cable and provides the ability for the pivot joint 91 to be extended after it bends. The pivot joint 91 is controlled by pivot joint motors 114 (only one shown). Pivot joint 93 operates in a similar fashion with its own cables, springs and pivot joint motors, as illustrated in FIG. 5.

In an example embodiment the pivot joints 91, 93 includes a clevis arrangement wherein a pin is positioned through each blade of the clevis to allow for the pivot joint 91, 93 to pivot. One side of the pivot joint 91, 93 may include a 45 degree chamfer to allow for a greater bend by the pivot joint.

As best illustrated in FIG. 4, the manipulator member 76 is also configured to move translationally, that is to extend and retract its distal end from the channels 72 to an exterior of the casing 70. To accomplish this, referring to FIG. 5, the flex shaft 94 is coupled to a translation motor 100 by way of a conventional screw and ball nut combination 102. Activation of the translation motor 100 will therefore effect translational movement to extend and retract the manipulator member 76 from and back to the casing 70 (FIG. 4).

The manipulator member 76 is also configured to effect axial rotation. A rotation motor 104 is coupled to a gear 106 which engages the flex shaft 94. Activation of the rotation motor 104 will effect rotation of the flex shaft 94, and subsequently the gripper 84.

Thus, in some example embodiments, the manipulator member 76 is configured for movement in at least four degrees of freedom (e.g., two rotational, axial rotational, and translational). A similar configuration may be used for the other manipulator members 74, 78, 80.

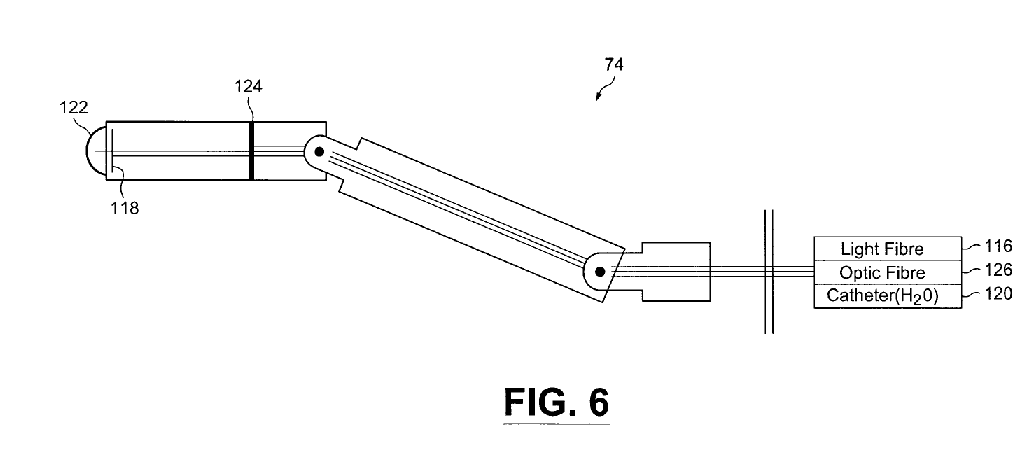

FIG. 6 shows the manipulator member 74 having a camera device 82 mounted thereon to be used as a detector. The camera device 82 includes a Charge-Coupled Device (CCD) 122 coupled to an optical fibre 126 for receiving of images and for transmitting to the display screen 42 (FIG. 1). In an example embodiment a 1.8 mm CCD is used. The camera view provided by the CCD 122 may be a traditional laparoscopic view, i.e., a “God's eye view” of the surgical site. A light ring 124 powered by an optical fibre 116 is also mounted to illuminate the surgical site. A water irrigation member 118 is supplied water by a catheter 120 and is used to clean the lens of the CCD 122 (the same member 118 may also be used for suction, as appropriate).

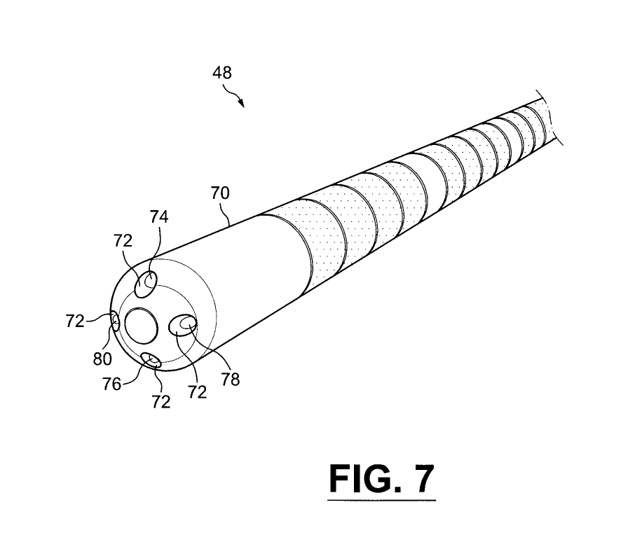

Reference is now made to FIGS. 7 to 9, which illustrate the end effector mechanism 48 in operation. As shown in FIG. 7, the manipulator members 74, 76, 78, 80 are shown normally nested within each of the channels 72 of the casing 70, and positioned adjacent to the exterior of the casing 70 to readily extend when needed. For example, the endoscopic device 20 may be navigated within the patient 14 to a desired surgical site. After which, referring to FIG. 8, the manipulator members 74, 76, 78, 80 may be extended and protruded from the channels to the exterior of the casing 70. Referring to FIG. 9, when in use, the manipulator segments of manipulator members 74, 76, 78, 80 are pivoted with respect to an adjacent manipulator segment. This provides the end effector mechanism 48 with additional degrees of freedom to manipulate the surgical site. The surgical end effector tools (e.g. the gripper 84) and camera device 82 may thereafter be controlled to be used on the surgical site. Once the task at the surgical site is completed, the manipulator members 74, 76, 78, 80 may be retracted to the nesting position within the casing 70, and may thereafter be navigated to another surgical site within the patient 14.

Referring now to FIGS. 1 and 2, the workstation 22 includes a display screen 42 and left and right hand-activated controllers 44, 46 to allow the practitioner 12 to manipulate and control the endoscopic device 20. The work station 22 also includes a controller such as a computing device 60 having suitable image processing, control, and other software installed thereon for operation of the system 10 and the workstation 22. The particular configuration of the hand-activated controllers 44, 46 is dependent on the particular application of the system 10. In some embodiments, the hand-activated controllers 44, 46 include haptic control for providing force feedback to the user, which may for example be proportional to the force applied or received by the end effector mechanism 48. In such embodiments, a force sensor (not shown) may be located within the casing 70 or mounted at the end of the manipulator member 74.

In example embodiments, when the cross-sectional diameter of the casing 70 is 20 mm (or less), the end effector mechanism 84 is operable in at least a 40 mm×20 mm area inside the patient 14.

Referring to FIG. 4, other detectors and sensors may for example be housed within the casing 70 or mounted at the end of the manipulator member 74. For example, an accelerometer (not shown) or 6 degree-of-freedom (DOF) sensor (not shown) may be used for determining orientation of the end effector mechanism 48 the body of the patient 1. By using an accelerometer, roll angle with respect to gravity may be determined, allowing for the controller 60 to calculate the necessary pixel/frame shift or rotation to display the image being captured by the camera 82 onto the display screen 42 with the image correct such that gravity is always pointing down. A 6 DOF sensor can also be included to for example allow for true positional data (include roll angle) of the end effector mechanism 48 inside the body by using a magnetic field generator placed along side the patient 14. In such an embodiment, the casing 40 may be formed of rigid plastic so as not to affect the magnetic field (other rigid materials such as metal may be used in other embodiments).

The surgical end effector tools may be retrofitted from existing tools or from OEM (original equipment manufacturer) tools. Exemplary surgical end effector tools include standard 3 mm endoscopic tools, forceps, bi-polar cutters, ultrasonic grippers, cauterizing tools, suturing devices and the like. Such tools may require remote activation of the features such as pulling a guide wire to close a jaw, or an electrical cable to supply current to an ultrasonic or bi-polar cutter.

Although some example embodiments have been described in the context of robotic surgery, it can be appreciated that certain embodiments may be adapted to traditional surgical devices and techniques. For example, some aspects of the end effector mechanism could be configured for use with a manually inserted and controlled endoscope.

The surgical system may also be adapted as a training system for teaching health practitioners in performing endoscopic surgery using the hand-activated controllers.

In one aspect, there is provided an end effector mechanism for an endoscopic device, the endoscopic device having a generally tubular portion. The end effector mechanism includes a casing for mounting at an end of the generally tubular portion of the endoscopic device, the casing defining a cavity open to an exterior of the casing, a manipulator member having a proximal end and a distal end, the distal end being nested within the cavity of the casing and extendable to protrude from the cavity to the exterior of the casing, and a surgical end effector tool mounted at the distal end of the manipulator member.

In another aspect, there is provided a method of using an end effector mechanism for an endoscopic device, the endoscopic device having a generally tubular portion. The end effector mechanism includes a casing for mounting at an end of the generally tubular portion of the endoscopic device, the casing defining a cavity open to an exterior of the casing, a manipulator member having a proximal end and a distal end, the distal end being nested within the cavity of the casing, and a surgical end effector tool mounted at the distal end of the manipulator member. The method includes extending the distal end to protrude from the cavity to the exterior of the casing. The method may further include inserting the end effector mechanism into a single access point of a patient, and navigating the end effector mechanism to a surgical site.

The example embodiments described herein are intended to be illustrative, and various changes and modifications may be effected therein by one skilled in the art.

1-20. (canceled)

21. An end effector mechanism for an endoscopic device, the endoscopic device having a generally tubular portion, the end effector mechanism comprising: a casing for mounting at an end of the generally tubular portion of the endoscopic device, the casing defining a cavity open to an exterior of the casing; a manipulator member having a proximal end and a distal end, the distal end being nested within the cavity of the casing and extendable to protrude from the cavity to the exterior of the casing; and a surgical end effector tool mounted at the distal end of the manipulator member.

22. An end effector as claimed in claim 21, wherein the distal end is nested within the cavity adjacent to the exterior of the casing.

23. An end effector as claimed in claim 21, wherein the cavity is shaped to snuggly correspond to a cross-sectional shape of the manipulator member.

24. An end effector as claimed in claim 21, wherein the manipulator member includes a plurality of serially coupled manipulator segments ending with a last manipulator segment, each manipulator segment pivotally connected to an adjacent manipulator segment, the surgical end effector tool being mounted at the last manipulator segment.

25. An end effector as claimed in claim 24, wherein one manipulator segment of the plurality of serially coupled manipulator segments is axially rotatable with respect to an adjacent manipulator segment.

26. An end effector as claimed in claim 21, further comprising a mechanical control for mechanically controlling of the manipulator member.

27. An end effector as claimed in claim 26, wherein the mechanical control is located within the casing.

28. An end effector as claimed in claim 21, further comprising:
a second cavity defined within the casing and open to the exterior of the casing, the second cavity being defined separately from said cavity; a second manipulator member having a proximal end and a distal end, the distal end being nested within the second cavity of the casing and extendable to protrude from the second cavity to the exterior of the casing.

29. An end effector as claimed in claim 28, further comprising a detector mounted at the distal end of the second manipulator member.

30. An end effector as claimed in claim 28, further comprising a second surgical end effector tool mounted at the distal end of the second manipulator member.

31. An end effector as claimed in claim 21, wherein the cavity includes a channel.

32. A method of using an end effector mechanism for an endoscopic device, the endoscopic device having a generally tubular portion, the end effector mechanism including a casing for mounting at an end of the generally tubular portion of the endoscopic device, the casing defining a cavity open to an exterior of the casing, a manipulator member having a proximal end and a distal end, the distal end being nested within the cavity of the casing, and a surgical end effector tool mounted at the distal end of the manipulator member, the method comprising: extending the distal end to protrude from the cavity to the exterior of the casing.

33. A method as claimed in claim 32, further comprising inserting the end effector mechanism into a single access point of a patient.

34. A method as claimed in claim 32, further comprising, prior to extending of the distal end, navigating the end effector mechanism to a surgical site.

35. A method as claimed in claim 32, further comprising retracting the distal end to nesting within the cavity of the casing.

36. A method as claimed in claim 32, wherein the distal end is nested within the cavity adjacent to the exterior of the casing.

37. A method as claimed in claim 32, wherein the manipulator member includes a plurality of serially coupled manipulator segments ending with a last manipulator segment, each manipulator segment pivotally connected to an adjacent manipulator segment, the surgical end effector tool being mounted at the last manipulator segment.

38. A method as claimed in claim 37, wherein one manipulator segment of the plurality of serially coupled manipulator segments is axially rotatable with respect to an adjacent manipulator segment.

39. A method as claimed in claim 32, further comprising mechanically controlling of the manipulator member using a mechanical control.

40. A method as claimed in claim 39, wherein the mechanical control is located within the casing.
